(19) United States
(12) Patent Application Publication
(10) Pub. No.: US 2004/0241223 A1
(43) Pub. Date: Dec. 2, 2004

(54) Oral dosage forms for macromolecular drugs

(76) Inventor: David Wong, Milpitas, CA (US)

Correspondence Address: David W. Barman, 1101 NE 176 Terrace, North Miami Beach, FL, 33162, US

(21) Appl. No.: 10/445,693

(22) Filed: May 27, 2003

(51) Int. Cl. A61K 9/20

(52) U.S. Cl. 424/464

(57) ABSTRACT

Drug delivery systems and methods for administering macromolecular drugs to the buccal mucosa for transmucosal drug delivery are described. The drug delivery systems comprise a drug composition containing an effective amount of the macromolecular drugs with or without an effective amount of a permeation enhancer for enhancing permeation of the macromolecular drugs through the mucosa and means to prevent macromolecular drugs leaching to buccal cavity and adjust the local pH similar to the pKa of the macromolecular drugs. These systems comprise a device of determined physical form, such as tablets, patches, and troches.

### BACKGROUND OF THE INVENTION

This invention relates to compositions and methods for delivery of drugs, especially macromolecular drugs, to a warm-blooded animal by transmucosal administration and particularly through the buccal mucosa of the oral cavity.

Evaluating membranes of the oral cavity as sites of drug administration have been done. And, there are advantages of drug administration through buccal and sublingual membranes over other routes: rapid onset of action, allowance of higher blood levels, avoiding the first-pass effect of hepatic metabolism and exposure of the drug to gastrointestinal fluid (which contain peptidases). Because the dosage form is applied to the oral cavity, it is also easy to apply, to localize and to remove the drug.

The sublingual route has received more attention than the buccal route. It includes the membrane of the ventral surface of the tongue and the floor of the mouth while the buccal mucosa constitutes the lining of the cheek and lips. It is thinner than the buccal mucosa, thus drug permeability is greater for the sublingual tissue. As it is more permeable than buccal mucosa, it gives faster absorption and higher bioavailabilities of many drugs. It is also convenient, easily accessible, and generally well accepted. For example, it is the known route for administration of nitroglycerin, buprenorphine and nifedipine. However, it lacks an expanse of smooth and relatively immobile mucosa to attach a retentive delivery system; it may not be suitable for sustained-release dosage forms. On the other hand, buccal mucosa is more suitable to attachment of retentive delivery systems.

The ability of a molecule to permeate through the oral mucosa is released to its molecular size, lipid solubility and ionization. Molecules, with molecular weight less than 300 daltons, across the mucosa rapidly. However, as molecular weight increases, permeability decreases. Mucosa is more permeable to lipid-soluble compounds than non-lipid soluble molecules, which is related to their partition coefficients. The degree of ionization of the molecule which depends on pKa of the molecules and the pH at the membrane surface affects greatly on permeability of the molecules. Maximum absorption occurs when the molecule is unionized or neutral in electrical charge, permeability decreases when the molecule is ionized. Therefore, absorption through the oral mucosa of charged macromolecular drugs is challenging.

Substances assisting the molecule transport across biological membranes are known as penetration enhancers. Penetration enhancers can be divided into (a) chelators (e.g., EDTA, citric acid, salicylates), (b) surfactants (e.g., sodium dodecyl sulfate (SDS)), (c) non-surfactants (e.g., unsaturated cyclic ureas), (d) bile salts (e.g., sodium deoxycholate, sodium taurocholate), and (e) fatty acids (e.g., oleic acid, acylcarnitines, mono- and diglycerides).

The efficacy of enhancers in transporting molecules across membranes is positively correlated with the enhancer's hydrophobicity. For instance, the effectiveness of bile salts on enhancing the insulin absorption through nasal membrane is related to the hydrophobicity of the steroid structure of the bile salt: deoxycholate<chenodeoxycholate<cholate<ursodeoxycholate. Various mechanisms of the action of penetration enhancers have been suggested and they are (1) reducing the viscosity and/or elasticity of mucus layer, (2) facilitating transcellular transport by increasing the fluidity of the lipid bilayer of membranes, (3) facilitating paracellular transport by altering tight junctions across the epithelial cell layer, (4) overcoming enzymatic barriers, and (5) increasing the thermodynamic activity of the drugs.

Typically, oral adhesives consist of a drug and one or more hydrophilic polymers that can adhere to a wet mucous surface. These adhesives are formulated either to release drug locally or permit drug adsorption through the mucosa.

In addition to typical bilayer structure of oral transmucosal dosage form, the present invention also includes a buffering agent in the hydrophilic layer to achieve the local pH similar to the pKa of the macromolecular drug. Such that, the macromolecular drug becomes unionized or its net charge becomes zero to facilitate the drug transport to the circulation system of individuals.

### OBJECTS AND SUMMARY OF THE INVENTION

It is an object of the present invention to provide a dosage form and method for administering a macromolecular drug that allow easy accessibility to the site of administration.

It is also an object of the invention to provide a dosage form and method for administering a macromolecular drug that promotes high patient acceptance and compliance.

These and other objects are accomplished by providing a drug delivery system for trans-buccal delivery of a macromolecular drug to an individual's buccal mucosa comprising:

(a) a drug composition comprising an effective amount of a macromolecular drug and an effective amount of a permeation enhancer for enhancing permeation of a macromolecular drug through the buccal mucosa; and

(b) a functional hydrophilic composition, wherein the drug composition and the hydrophilic composition are combined in a single formulation.

The drug delivery system is preferably embodied in either a device of determined physical form, such as a tablet, patch, or troche. The preferred physical forms are bi-layer tablet, extruded film or patch.

The permeation enhancer is preferably a member selected from the group consisting of cell envelope disordering compounds, steroidal detergents, chelators, surfactants, non-surfactants, fatty acids, and mixtures thereof. A preferred cell-envelope disordering compound is a member selected from the group consisting of isopropyl myristate, methyl laurate, oleic acid, oleyl alcohol, glycerol monoleate, glycerol dioleate, glycerol trioleate, glycerol monostearate, glycerol monolaurate, propylene glycol monolaurate, sodium dodecyl sulfate, and sorbitan esters and mixtures thereof. A preferred bile salt is a steroidal detergent selected from the group consisting of natural and synthetic salts of cholanic acid and mixtures thereof.

A preferred tablet according to the invention comprises an adhesive drug layer comprising the drug, one or more permeation enhancer and one or more hydrophilic polymer. This adhesive drug layer has one surface adapted to contact a first tissue of the oral cavity and adhere thereto when wet and an opposing surface in contact with and adhering to an adjacent hydrophilic layer comprising a high molecular weight hydrophilic polymer. Preferably the hydrophilic polymer comprises at least one member selected from the group consisting of hydroxypropyl cellulose, hydroxypropyl methylcellulose, hydroxyethylcellulose, ethylcellulose, carboxymethyl cellulose, dextran, gaur-gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and mixtures thereof. It is also preferred that the adhesive layer additionally contain one or more members selected from the group consisting of fillers, tableting excipients, lubricants, flavors, and dyes and that the drug/enhancer layer additionally contain one or members selected from the group consisting of tableting excipients, fillers, flavors, taste-masking agents, dyes, stabilizers, enzyme inhibitors, and lubricants.

A method of delivering a macromolecular drug for transbuccal drug delivery to an individual's buccal mucosa comprises bringing the buccal mucosa into contact with a delivery system comprising:

(a) a drug composition comprising an effective amount of a macromolecular drug; and

(b) a functional hydrophilic composition, wherein the drug composition and the functional hydrophilic composition are combined in a single formulation;

and retaining said delivery system in contact with said mucosa for a time sufficient to delivery an effective amount of said macromolecular drug to said individual.

### DETAILED DESCRIPTION OF THE INVENTION

Before the present compositions and methods for buccal delivery of a macromolecular drug are disclosed and described, it is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, reference to a bilayer tablet containing macromolecular drugs includes a mixture of two or more of such macromolecular drugs, reference to “an adhesive” includes reference to one or more of such adhesives, and reference to “permeation enhancer” includes reference to a mixture of two or more of such permeation enhancers.

In describing and claiming the present invention, the following terminology will be used in accordance with the definitions set out below.

As used herein, “macromolecular drug” refers to nucleotides, macromolecular drugs and polysaccharides of any length.

As used herein, “permeation enhancer,” “chemical enhancer,” “penetration enhancer,” and the like shall be inclusive of all enhancers that increase the flux of a drug, permeant, or other molecule across the mucosa. In other words, all cell envelope disordering compounds, solvents, steroidal detergents, chelators, surfactants, non-surfactants, fatty acids, bile salts, and any other chemical enhancement agents are intended to be included.

The flux of a drug across the mucosa can be increased by changing either the gradient for diffusion or the diffusion coefficient. Flux may be enhanced by the use of so-called permeation or chemical or penetration enhancers.

Penetration enhancers are comprised of two primary groups: “cell-envelope disordering compounds and solvents” and “binary systems containing both cell-envelope disordering compounds and solvents”. As discussed above, other groups of permeation enhancers are steroidal detergents, bile salts, chelators, surfactants, non-surfactants, and fatty acids.

Cell envelope disordering compounds are useful in topical pharmaceutical preparations and can be used for drug delivery through the skin or mucosa. These compounds assist dermal penetration by disordering the lipid structure of the stratum corneum cell-envelopes.

A list of such compounds can be found in European Patent Application 43,738, published Jun. 13, 1982 incorporated herein by reference. Cell envelope disordering compounds can be used in this invention. The cell envelope disordering compounds are represented by the following formula:

R—X

wherein R is a straight-chain alkyl of about 7 to 16 carbon atoms, a non-terminal alkenyl of about 7 to 22 carbon atoms, or a branched-chain alkyl of from about 13 to 22 carbon atoms, and X is —OH, —COOCH.sub.3, —COOC.sub.2H.sub.5, —OCOCH.sub.3, —SOCH.sub.3, —P(CH.sub.3).sub.2 O, —COOC.sub.2H.sub.40C.sub.2H.sub.40H, —COOCH(CHOH).sub.40H.sub.20H, —COOCH.sub.2 CHOHCH3, —COOCH.sub.2 CH(OR″)CH.sub.20R″, —(OCH.sub.2 CH.sub.2).sub.m OH, —COOR′, or—CONR′.sub.2 where R′ is —H, —CH.sub.3, —C.sub.2H.sub.5, —C.sub.3H.sub.7 or—C.sub.2H.sub.40H; R″ is —H, or a non-terminal alkenyl of about 7 to 22 carbon atoms; and m is 2-6; provided that when R″ is an alkenyl and X is —OH or —COOH, at least one double bond is in the cis-configuration.

Solvents include diols (e.g. propylene glycol, glycerol), alcohols (e.g. ethanol, propanol, higher alcohol), water, dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted-alkyl-azacycloalkyl-2-ones (azones) and the like.

An excellent summary of prior art and background information for certain types of binary systems for permeant enhancement can be found in U.S. Pat. No. 4,537,776, Cooper, issued Aug. 27, 1985 incorporated herein by reference. Details about using selected diols as solvents and a broad category of cell-envelope disordering compounds for delivery of lipophilic drugs are described in European Patent 43,738 incorporated herein by reference. Example of application of binary system for enhancing metoclopramide penetration can be found in U.S. Pat. No. 4,605,670. Examples of combinations of enhancers are disclosed in U.S. Pat. No. 4,973,468.

Other penetration enhancers include DMSO or aqueous solutions of DMSO, the azones, and bile salts. Bile salts here mean the steroidal detergents that are either unionized or ionized forms of cholanic acids such as the salt or unionized forms of cholic and deoxycholic acid.

As used herein, “transmucosal,” “transbuccal,” and similar terms mean passage of a macromolecular drug into and through the buccal mucosa to achieve effective therapeutic blood levels.

As used herein, “effective amount” means an amount of a macromolecular drug that is nontoxic but sufficient to perform a beneficial pharmacological effect on individuals. An effective amount of a penetration enhancer, as used herein, means an amount used to provide the selected increase in mucosal permeability and, correspondingly, the rate of administration, and amount of drug delivered.

As used herein, “hydrophilic polymer” means a natural or synthetic polymer which, by the hydrophilic designation, can be either water-soluble or swellable and which are compatible with the enhancers and macromolecular drugs. It also possesses of adhesive function for adhering the dosage forms to the mucous tissues of the oral cavity. The hydrophilic polymer comprises hydroxypropyl cellulose, hydroxypropyl methylcellulose, hydroxyl ethylcellulose, ethylcellulose, carboxymethyl cellulose, dextran, gaur gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and mixtures thereof, and the like.

By “system”, “drug delivery system”, “transmucosal delivery system” or the like is meant a unit dosage form of a drug composition and a functional hydrophilic composition. The drug composition comprising penetration enhancers, drug and a hydrophilic polymer, which adheres to the oral mucosa, which the functional hydrophilic composition prevents any macromolecular drugs “leak” to the oral cavity and it also maintains the local pH close to the pKa of the macromolecular drugs. Such systems can be either a patch, tablet or troche to be held against the buccal mucosa to continuous drug delivery to the systemic circulation.

The term “troche” includes pastille, lozenge, morsulus, rotula, trochiscus, and the like. A matrix patch contains the drug, permeation enhancer, and other optional ingredients suspended or dispersed in an adhesive layer. A reservoir patch contains the drug, permeation enhancer, and other optional ingredients in a reservoir, which can be in liquid form, or the liquid can be gelled or thickened by an agent such as mineral oil, petroleum jelly and various aqueous gelling agents and hydrophilic polymers. Such a reservoir or matrix patch is adhesive to the buccal mucosa and is attached by a functional hydrophilic layer. The method of application of the present invention can vary within limits, but necessarily involves applying the selected drug composition to the buccal mucosa such that drug delivery is initiated and continues for a period of time sufficient to provide the beneficial effect.

Bilayer tablets can be made by classical bilayer tablet compression techniques on a suitable press. The bilayer tablet consists of an adhesive drug layer and a functional hydrophilic layer, which can be of a different color to distinguish the layers for purposes of application. The identification of the drug-containing, adhesive layer facilitates application by the patient and prevents incidental adhesion of the functional hydrophilic layer to the oral tissues. The adhesive drug layer is first prepared by either dry-mixing the ingredients and compressing them into a tablet or by wet granulating the ingredient mixture and then compressing according to accepted pharmaceutical techniques. In general, the ingredients in the adhesive drug formulation include the drug, flavors, taste-masking agents, stabilizers, enzyme inhibitors, fillers, diluents, binders, penetration enhancers, hydrophilic polymers, lubricants, flavors, dyes, and the like. The functional hydrophilic layer is prepared by intimately admixing high-molecular weight hydrophilic polymers, diluents, fillers, dyes, lubricants, and the like. This can be formulated as a dry mix or accomplished by conventional wet granulation and screening techniques followed by drying. In either event, the blended drug-containing layer ingredients are then placed on top of the partially compressed adhesive layer and both layers are then compressed. A person of ordinary skill in the art will recognize that the instant tablet can also be manufactured by making the drug-containing layer first and then the adhesive layer.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof that do not depart from the spirit and scope of the invention.

I claim:

1. A drug delivery system for trans-buccal delivery of macromolecular drugs to an individual's buccal or sublingual mucosa comprising:
(a) a drug composition comprising a polymer and an effective amount of one or more macromolecular drugs; and
(b) a functional hydrophilic composition comprising one or more water-soluble polymer.

2. The drug delivery system of claim 1 wherein said system comprises a device of determined physical form.

3. The drug delivery system of claim 2 wherein said device of determined physical form is a member selected from the group consisting of a patch, a tablet, a troche and a extruded film and wherein said the drug composition is an adhesive.

4. The drug delivery system of claim 3 optionally includes one or more permeation enhancer.

5. The drug delivery system of claim 4 wherein said permeation enhancer is a member selected from the group consisting of cell envelope disordering compounds, solvents, steroidal detergents, chelators, surfactants, non-surfactants, fatty acids, and mixtures thereof.

6. The drug delivery system of claim 1 wherein said tablet comprises an adhesive drug layer comprising a macromolecular drug, a permeation enhancer and a hydrophilic polymer having one surface adapted to contact a first tissue of the oral cavity and adhere thereto when wet and an opposing surface in contact with and adhering to a functional hydrophilic layer comprising a high molecular weight hydrophilic polymer and a buffering agent.

7. The drug delivery system of claim 6 wherein said hydrophilic polymer comprises at least one member selected from the group consisting of hydroxypropyl cellulose, hydroxypropyl methylcellulose, hydroxyethylcellulose, ethylcellulose, carboxymethyl cellulose, dextran, gaur-gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and mixtures thereof.

8. The drug delivery system of claim 6 wherein said functional hydrophilic layer additionally contains at least one member selected from the group consisting of fillers, tableting excipients, buffering agents, hydrophilic polymers, lubricants, flavors, and dyes and wherein said drug layer additionally contains at least one member selected from the group consisting of tableting excipients, hydrophilic polymers, fillers, flavors, taste-masking agents, dyes, stabilizers, enzyme inhibitors, and lubricants.

9. The drug delivery system of claim 6 wherein said buffering agent comprising an acid and/or a base or a mixture of an acid and a base to achieve the pH of the delivery site similar to the pKa of the macromolecular drug.
